(19) United States
(12) Patent Application Publication
(10) Pub. No.: US 2013/0039998 A1
(43) Pub. Date: Feb. 14, 2013

(54) COMPOSITIONS OF MODULATORS OF THE WNT/BETA-CATENIN PATHWAY AND BENZAMIDE AND/OR HYDROXAMIC ACID DERIVATIVES TO TREAT BIPOLAR DISORDER

(71) Applicants: Daniel Fass, Winchester, MA (US); Stephen Haggarty, Dorchester, MA (US); Edward Scolnick, Wayland, MA (US); Randall T. Moon, Kenmore, WA (US); Travis L. Biechele, Seattle, WA (US); Nathan D. Camp, Seattle, WA (US)

(72) Inventors: Daniel Fass, Winchester, MA (US); Stephen Haggarty, Dorchester, MA (US); Edward Scolnick, Wayland, MA (US); Randall T. Moon, Kenmore, WA (US); Travis L. Biechele, Seattle, WA (US); Nathan D. Camp, Seattle, WA (US)

(73) Assignees: UNIVERSITY OF WASHINGTON, Seattle, WA (US); MASSACHUSETTS INSTITUTE OF TECHNOLOGY, Cambridge, MA (US)

(21) Appl. No.: 13/141,747

(22) PCT Filed: Dec. 23, 2009
(86) PCT No.: PCT/US2009/069472

§ 371 Date: Feb. 14, 2012

Related U.S. Application Data
(60) Provisional application No. 61/140,728, filed on Dec. 24, 2008.

(51) Int. Cl. A61K 31/167; A61K 31/4406; A61P 25/00; A61K 31/513; A61K 33/14; A61K 33/00; A61K 31/4045; A61K 31/194

(52) U.S. Cl. 424/677; 514/616; 514/415; 514/357; 514/574; 514/274; 424/715

(57) ABSTRACT

The present invention is directed to a composition comprising a modulator of the Wnt/β-catenin pathway, such as a GSK-3 inhibitor, or a pharmaceutically acceptable salt thereof either with a benzamide derivative or a pharmaceutically acceptable salt thereof or with a hydroxamic acid or a pharmaceutically acceptable salt thereof. The present invention is also directed to a method of treating bipolar disorder in a subject by administering either a benzamide derivative or a hydroxamic acid to the subject under conditions effective to treat a bipolar disorder. A modulator of the Wnt/β-catenin pathway, such as a GSK-3 inhibitor, or a pharmaceutically acceptable salt thereof may also be administered to a subject.

### FIELD OF THE INVENTION

This invention relates to compositions of modulators of the Wnt/13-catenin pathway, such as GSK-3 inhibitors, and a benzamide and/or a hydroxamic acid derivative to treat bipolar disorder.

### BACKGROUND OF THE INVENTION

Bipolar disorder is a psychiatric condition defined as recurrent episodes of significant disturbance in mood. These disturbances can occur on a spectrum that ranges from debilitating depression to unbridled mania. Individuals suffering from bipolar disorder typically experience fluid states of mania, hypomania, or what is referred to as a mixed state in conjunction with depressive episodes. These clinical states typically alternate with a normal range of mood. The disorder has been subdivided into bipolar I, bipolar II and cyclothymia, with both bipolar I and bipolar II potentially presenting with rapid cycling.

Bipolar disorder is a cyclic illness where people periodically exhibit elevated (manic) and depressive episodes. Most people will experience a number of episodes, averaging 0.4 to 0.7 a year with each lasting 3 to 6 months. Late adolescence and early adulthood are peak years for the onset of the illness. These are critical periods in a young adult's social and vocational development, and they can be severely disrupted by disease onset.

Bipolar disorder cannot be cured; instead the emphasis of treatment is on effective management of acute episodes and prevention of further episodes by use of pharmacological and psychotherapeutic techniques.

The mainstay of treatment is a mood stabilizer medication; these comprise several unrelated compounds which have been shown to be effective in preventing relapses of manic, or in the one case, depressive episodes. The first known and “gold standard” mood stabilizer is lithium, while almost as widely used is sodium valproate, originally used as an anticonvulsant. Other anticonvulsants used in bipolar disorder include carbamazepine, reportedly more effective in rapid cycling bipolar disorder, and lamotrigine, which is the first one to be shown to be of benefit in bipolar depression.

Treatment of the agitation in acute manic episodes has often required the use of antipsychotic medications, such as Quetiapine, Olanzapine, and Chlorpromazine.

The use of antidepressants in bipolar disorder has been debated, with some studies reporting a worse outcome with their use triggering manic, hippomanic or mixed episodes, especially if no mood stabilizer is used. However, most mood stabilizers are of limited effectiveness in depressive episodes.

The present invention is directed to overcoming these and other deficiencies in the art.

### SUMMARY OF THE INVENTION

One aspect of the present invention is directed to a composition comprising a modulator of the Wnt/β-catenin pathway or a pharmaceutically acceptable salt thereof and a benzamide derivative, said benzamide derivative having the formula:

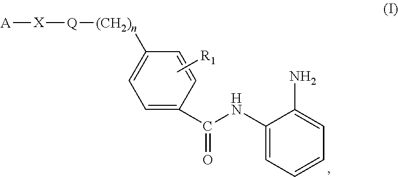

where

A is H, a substituted or unsubstituted single-, fused- or multiple-ring aryl or heterocyclic ring systems, including saturated and unsaturated N-heterocycles, saturated and unsaturated S-heterocycles, and saturated and unsaturated O-heterocycles, saturated or unsaturated cyclic hydrocarbons, saturated or unsaturated mixed heterocycles;

X is absent,

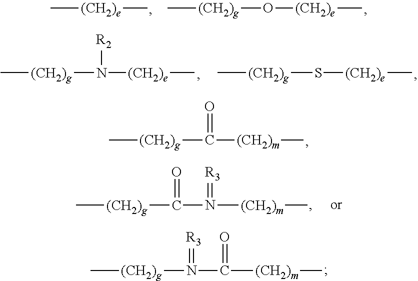

Q is

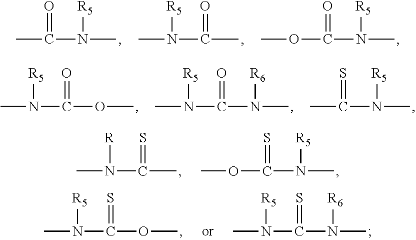

e is an integer from 1 to 4;

g is an integer from 0 to 4;

m is an integer from 0 to 4;

R₁ is H, halogen, hydroxyl, amino, nitro, cyano, C₁-C₄ alkyl, C₁-C₄ alkoxy, C₁-C₄ aminoalkyl, C₁-C₄ alkylamino, C₁-C₄ acyl, C₁-C₄ acylamino, C₁-C₄ alkylthio, C₁-C₄ perfluoroalkyl, C₁-C₄ perfluoroalkoxy, carboxyl, C₁-C₄ alkoxycarbonyl, aryl, or heterocycle or heteroaryl containing 1 to 5 heteroatoms selected from the group consisting of oxygen, nitrogen, and sulfur;

R₂ is H, substituted or unsubstituted C₁-C₄ alkyl, or

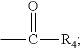

R₃ is H, or substituted or unsubstituted C₁-C₄ alkyl;

R₄ is H, substituted or unsubstituted C₁-C₄ alkyl, substituted or unsubstituted C₁-C₄ perfluoroalkyl, substituted or unsubstituted phenyl, or substituted or unsubstituted heterocycle;

R₅ and R₆ are each independently H, or substituted or unsubstituted C₁-C₄ alkyl; and

n is an integer from 0 to 4, or a pharmaceutical salt thereof.

Another aspect of the present invention is directed to a method of treating a bipolar disorder in a subject. The method includes administering the above benzamide derivative or a pharmaceutically acceptable salt thereof under conditions effective to treat a bipolar disorder. A modulator of the Wnt/β-catenin pathway or a pharmaceutically acceptable salt thereof may optionally be administered together with the benzamide derivative.

Still another aspect of the present invention is directed to a composition comprising a modulator of the Wnt/β-catenin pathway, such as an inhibitor of GSK-3 activity, or a pharmaceutically acceptable salt thereof and a hydroxamic acid derivative, said hydroxamic acid derivative having the formula:

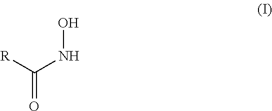

where R is C₂-C₆ alkyl, optionally substituted with substituents selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃; R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl; R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄; R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof,

or

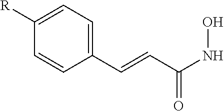

wherein,

R is selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;

R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;

R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;

R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof.

Another aspect of the present invention is directed to a method of treating a bipolar disorder in a subject. The method includes administering the above hydroxamic acid derivative or a pharmaceutically acceptable salt thereof under conditions effective to treat a bipolar disorder. A modulator of the Wnt/β-catenin pathway, such as an inhibitor of GSK-3 activity, or a pharmaceutically acceptable salt thereof may optionally be administered together with the hydroxamic acid derivative.

Methods of enhancing Wnt/β-catenin signaling are currently not available. The present invention overcomes this limitation by providing classes of chemicals that can enhance Wnt/β-catenin signaling. These chemicals may be relevant therapeutically in a number of settings. For example, there are currently no approved drugs that are known to enhance the activity of the mood stabilizer lithium. Lithium has both anti-manic and anti-depressant activity in humans. Lithium is also being investigated in neurodegenerative disorders, including Alzheimer's disease, and in fragile X syndrome. It is expected that the benzamide and hydroxamic acids described herein will enhance the clinical benefit of lithium either by reducing the dose of lithium needed (time or amount) or speed the onset of its clinical benefit.

### BRIEF DESCRIPTION OF THE DRAWINGS

FIGS. 1A-B show monitoring of Wnt/β-catenin signaling using beta-catenin activated reporter (BAR) system. FIG. 1A is a schematic of the pBARLS reporter of TCF/LEF transcription. FIG. 1B is a graph showing activation of pBAR reporter in HEK-293T cells (human) and HT22 cells (mouse) by Wnt3a in both cell lines, but only by lithium in HEK-293T.

FIGS. 2 A-B show the activation of TCF/LEF transcription by MS-275, CI-994, and SAHA. FIG. 2A is a graph showing results from the pBARLS reporter in HEK293T cells. 24 hr treatment of cells with lithium (10 mM), MS-275 (25 μM), CI-994 (25 μM), and SAHA (25 μM) activate transcription on their own (SAHA only ˜2.5-fold) and show strong synergy with lithium treatment. FIG. 2B is a graph showing results from pBARLS reporter in HT22 cells. 24 hr treatment of cells with lithium (10 mM), CI-994 (25 μM) and SAHA (25 μM) is unable to activate transcription, but MS-275 (25 μM) is capable of activating transcription on its own. In the presence of lithium, all three compounds show strong synergistic activation of transcription.

FIGS. 3 A-B show structural classes of synergistic activators of TCF/LEF transcription. FIG. 3A depicts structural formulae of the carboxylic acid valproate, the benzamide MS-275, and the hydroxamate panobinostat and their half-effective concentration for activation of reporter in the presence of lithium in HEK-293T. FIG. 3B is a graph showing results from pBARLS reporter in HEK293T cells with 24 hr treatment of cells with lithium (10 mM), valproate, MS-275, and panobinostat, showing activation on their own and synergistic activation in the presence of lithium.

FIG. 4 shows structural formulas of exemplary compounds and their division into two structural classes.

### DETAILED DESCRIPTION OF THE INVENTION

One aspect of the present invention is directed to a composition comprising a modulator of the Wnt/β-catenin pathway or a pharmaceutically acceptable salt thereof and a benzamide derivative, said benzamide derivative having the formula:

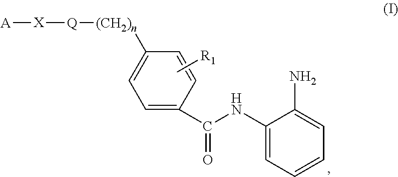

where

A is H, a substituted or unsubstituted single-, fused- or multiple-ring aryl or heterocyclic ring systems, including saturated and unsaturated N-heterocycles, saturated and unsaturated S-heterocycles, and saturated and unsaturated O-heterocycles, saturated or unsaturated cyclic hydrocarbons, saturated or unsaturated mixed heterocycles;

X is absent,

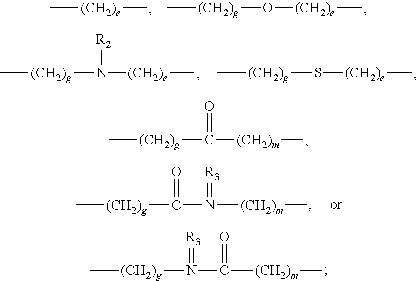

Q is

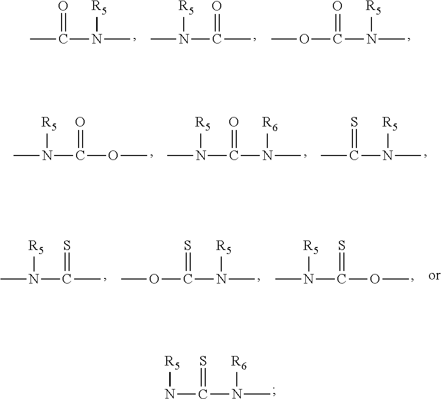

e is an integer from 1 to 4;

g is an integer from 0 to 4;

m is an integer from 0 to 4;

R₁ is H, halogen, hydroxyl, amino, nitro, cyano, C₁-C₄ alkyl, C₁-C₄ alkoxy, C₁-C₄ aminoalkyl, C₁-C₄ alkylamino, C₁-C₄ acyl, C₁-C₄ acylamino, C₁-C₄ alkylthio, C₁-C₄ perfluoroalkyl, C₁-C₄ perfluoroalkoxy, carboxyl, C₁-C₄ alkoxycarbonyl, aryl, or heterocycle or heteroaryl containing 1 to 5 heteroatoms selected from the group consisting of oxygen, nitrogen, and sulfur;

R₂ is H, substituted or unsubstituted C₁-C₄ alkyl, or

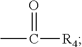

R₃ is H, or substituted or unsubstituted C₁-C₄ alkyl;

R₄ is H, substituted or unsubstituted C₁-C₄ alkyl, substituted or unsubstituted C₁-C₄ perfluoroalkyl, substituted or unsubstituted phenyl, or substituted or unsubstituted heterocycle;

R₅ and R₆ are each independently H, or substituted or unsubstituted C₁-C₄ alkyl; and

n is an integer from 0 to 4, or a pharmaceutical salt thereof.

In certain embodiments, the benzamide derivative has the formula:

where

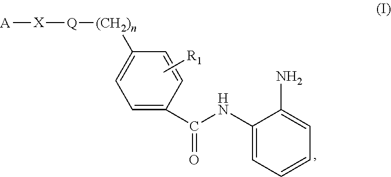

A is H or an unsaturated N-heterocycle;

X is absent,

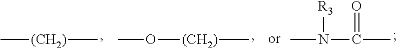

Q is

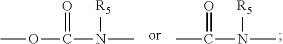

R₁, R₃, and R₅ are each H; and

n is 1, or a pharmaceutical salt thereof.

In a preferred embodiment, the benzamide derivative has the formula:

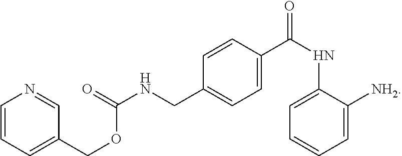 [SMILES: Nc1ccccc1NC(=O)c1ccc(CNC(=O)OCc2cccnc2)cc1]

In another preferred embodiment, the benzamide derivative has the formula:

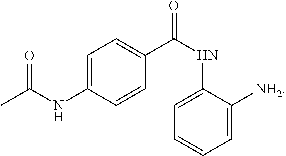 [SMILES: CC(=O)Nc1ccc(C(=O)Nc2ccccc2N)cc1]

Suitable modulators of the Wnt/β-catenin pathway may be lithium or a pharmaceutically acceptable salt thereof, or an inhibitor of a GSK-3 kinase or a pharmaceutically acceptable salt thereof. In a preferred embodiment, the GSK-3 kinase is GSK-3 β.

The lithium salt can be selected from the group consisting of lithium chloride, lithium citrate, lithium carbonate, lithium orotate, and mixtures thereof. In a preferred embodiment, the lithium salt is lithium chloride.

Modulation of the Wnt/β-catenin pathway may be done by a β-catenin signal-promoting agent. “β-catenin signal-promoting agent” refers to agonists or antagonists of positive or negative signaling molecules, respectively, of β-catenin signaling, e.g., any agent that activates β-catenin signaling through inhibition of GSK-3 in the presence or absence of Wnt signaling. For example, activation of β-catenin signaling in the absence of Wnt signaling can occur by activation of integrin linked kinase, activation of p53 leading to activation of Siah1, or activation of FGF signaling. “β-catenin signal-promoting agent” further refers to a signaling molecule that activates β-catenin target genes and is achieved by inhibition of GSK-3 that can have therapeutic potential. “β-catenin signal-promoting agent” further refers to any signaling molecule that activates β-catenin target genes independent of GSK-3 that can have therapeutic potential. Activation of β-catenin target genes without inhibiting GSK-3 can be achieved by inhibition (for example, by drug therapy, RNAi therapy or gene therapy) of any inhibitor of β-catenin function, including, but not limited to, APC, Axin, Chibby, ICAT, Groucho, and CtBP.

Modulation of the Wnt/β-catenin pathway may be done by a Wnt signal- or β-catenin signal-promoting agent. “Wnt signal- or β-catenin signal-promoting agent” refers to one or more of the following: a nucleic acid comprising a nucleotide sequence that encodes a Wnt polypeptide, a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, Notch signaling or Hedgehog signaling, a small organic molecule that inhibits the expression or activity of a Wnt, β-catenin, Notch or Hedgehog antagonist, an antisense oligonucleotide that inhibits expression of a Wnt, β-catenin, Notch or Hedgehog antagonist, a ribozyme that inhibits expression of a Wnt, β-catenin, Notch or Hedgehog antagonist, an RNAi construct, siRNA, or shRNA that inhibits expression of a Wnt, β-catenin, Notch or Hedgehog antagonist, an antibody that binds to and inhibits the activity of a Wnt, β-catenin, Notch or Hedgehog antagonist, a nucleic acid comprising a nucleotide sequence that encodes a β-catenin polypeptide, a polypeptide comprising an amino acid sequence of a β-catenin polypeptide, a nucleic acid comprising a nucleotide sequence that encodes a Lef-1 polypeptide, a polypeptide comprising an amino acid sequence of a Lef-1 polypeptide.

“Wnt/β-catenin signal-promoting agent” refers to one or more of the following: a nucleic acid comprising a nucleotide sequence that encodes a dominant negative GSK-3, GSK3α, or GSK3β polypeptide, a polypeptide comprising an amino acid sequence of a dominant negative GSK-3, GSK3α, or GSK3β polypeptide, a small organic molecule that binds to and inhibits the expression or activity of GSK-3, GSK3α, or GSK3β, an RNAi construct, siRNA, or shRNA that binds to and inhibits the expression and/or activity of GSK-3, GSK3α, or GSK3β, an antisense oligonucleotide that binds to and inhibits the expression and/or activity of GSK-3, GSK3α, or GSK3β, an antibody that binds to and inhibits the expression and/or activity of GSK-3, GSK3α, or GSK3β, a ribozyme that binds to and inhibits the expression of GSK-3, GSK3α, or GSK3β, and any GSK-3 independent reagent that activates β-catenin target genes similar in effect to GSK-3 inhibition.

Exemplary Wnt/β-catenin signal-, Notch signal- or Hedgehog signal-promoting agents include, but are not limited to, LiCl or other GSK-3 inhibitors, as exemplified in U.S. Pat. Nos. 6,057,117 and 6,608,063; and U.S. Patent Publication Nos. 2004/0092535 and 2004/0209878, which are hereby incorporated by reference in their entirety; ATP-competitive, selective GSK-3 inhibitors CHIR-911 and CHIR-837 (also referred to as CT-99021 and CT-98023 respectively) Chiron Corporation (Emeryville, Calif.). These inhibitors were purified >95% by high-performance liquid chromatography. CHIR-911 was formulated in 10% captisol solution for administration in vivo by intraperitoneal injection, with a half-maximal effective concentration [EC₅₀] of 766 nM and >10,000 fold selectivity for GSK-3. See Ring et al., Diabetes 52:588-595 (2003), which is hereby incorporated by reference in its entirety. CHIR-837 was formulated in DMSO for in vitro use, with an EC₅₀ of 375 nM and >5,000 fold selectivity for GSK-3. See Cline et al., Diabetes 51:2903-2910 (2002), which is hereby incorporated by reference in its entirety. GSK-3 inhibitor CHIR025, see Kelley, S., Exp. Neurol., 188:378-386 (2004), which is hereby incorporated by reference in its entirety.

Further exemplary Wnt/β-catenin signal-promoting agents include, but are not limited to, GSK-3 inhibitors such as SB-216763 and SB-415286, developed by Glaxo Smith Kline (see Eldar-Finkelman, et al., TRENDS in Molecular Medicine 8(3):126-132 (2002) and Patel et al., Biochemical Society Transactions 32(5):803-808 (2004), which are hereby incorporated by reference in their entirety). Calbiochem® Alzheimer's and Other Neurodegenerative Disease Research Tools, EMD Biosciences, Inc. San Diego, Calif., pgs 1-32 (2003), which is hereby incorporated by reference in its entirety, additionally discloses aloisine A, Aloisine RP106, alsterpaullone, a thiadiazolidinone analog, a 2-thio[1,3,4]-oxadiazole-pyridyl derivative, an oxothiadiazolidine-3-thione analog, indirubin-3′-monoxime, 5-iodo-indirubin-3′-monoxime, indirubin-3′-monoxime-5-sulphonic acid, and kanpaullone as small molecule GSK-3 inhibitors and GSK-3 peptide inhibitors H-KEAPPAPPQSpP-NH₂ and Myr-N-GKEAPPAPPQSpP-NH₂. Kulkarni et al., J. Bone and Mineral Res., 21(6):910-920 (2006), which is hereby incorporated by reference in its entirety, identifies 603281-31-8 as another small molecule GSK-3 inhibitor. Still further exemplary GSK-3 inhibitors include, but are not limited to, compound 603281-31-8 developed by Lilly (see Kukarni, N., et al., J. Bone Miner. Res., 21:910-920 (2006), which is hereby incorporated by reference in its entirety), the FRATide peptide (see Bax, B., et al. Structure, 9:1143-1152 (2001), which is hereby incorporated by reference in its entirety), 6-bromoindirubin-3′ oxime (see Parkitna, J., et al., JPET DOI: 10.1124/jpet.106.107581 (2006), which is hereby incorporated by reference in its entirety), and retinoic acid (see Eisinger, A., et al., J. Biol. Chem., 282(40):29394-29400 (2007), which is hereby incorporated by reference in its entirety).

Other GSK-3 inhibitors include zinc, Akt, PKC, PKA, p90RSK, thymoleptics (e.g. valproate, MAOIs, fluoxetine, imipramine, clozapine, risperidone, and haloperidol), estrogen, L803-mts, and AR-A014418. See Gould, T., et al., Neuropsychopharm, 1-15 (2005), which is hereby incorporated by reference in its entirety. Other stimulators of 5-HT_{1A} receptors that inhibit GSK-3 are described in Beaulieu, J., Int'l J. Neuropsychopharm., 1-4 (2006), which is hereby incorporated by reference in its entirety.

In certain embodiments, the composition includes a pharmaceutically acceptable excipient or carrier.

Another aspect of the present invention is directed to a method of treating a bipolar disorder in a subject. The method includes administering the above benzamide derivative or a pharmaceutically acceptable salt thereof under conditions effective to treat a bipolar disorder. A modulator of the Wnt/β-catenin pathway or a pharmaceutically acceptable salt thereof may optionally be administered together with the benzamide derivative.

The modulator of the Wnt/β-catenin pathway or a pharmaceutically acceptable salt thereof and the benzamide derivative or a pharmaceutically acceptable salt thereof are in the form substantially as those described above.

The method may include selecting a subject with bipolar disorder and administering the benzamide derivative or a pharmaceutically acceptable salt thereof and, optionally, the modulator of the Wnt/β-catenin pathway or a pharmaceutically acceptable salt thereof.

Still another aspect of the present invention is directed to a composition comprising a modulator of the Wnt/β-catenin pathway, such as an inhibitor of GSK-3 activity, or a pharmaceutically acceptable salt thereof and a hydroxamic acid derivative, said hydroxamic acid derivative having the formula:

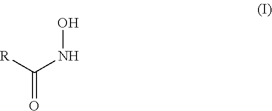

where R is C₂-C₆ alkyl, optionally substituted with substituents selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃; R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl; R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄; R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof,

or

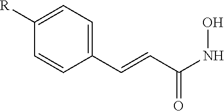

wherein,

R is selected from the group consisting of hydroxy, halogen, —NH₂₅—NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;

R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;

R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;

R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof.

In a preferred embodiment, the hydroxamic acid derivative has the formula:

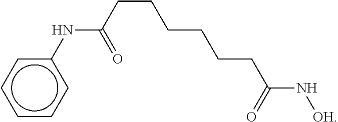

In another preferred embodiment, the hydroxamic acid derivative has the formula:

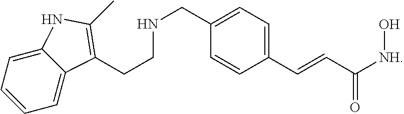 [SMILES: Cc1[nH]c2ccccc2c1CCNCc1ccc(/C=C/C(=O)NO)cc1]

Suitable modulators of GSK-3 activity are described above.

In certain embodiments, the composition includes a pharmaceutically acceptable excipient or carrier.

Another aspect of the present invention is directed to a method of treating a bipolar disorder in a subject. The method includes administering the above hydroxamic acid derivative or a pharmaceutically acceptable salt thereof under conditions effective to treat a bipolar disorder. A modulator of the Wnt/β-catenin pathway, such as an inhibitor of GSK-3 activity, or a pharmaceutically acceptable salt thereof may optionally be administered together with the hydroxamic acid derivative.

The method may include selecting a subject with bipolar disorder and administering the hydroxamic acid derivative or a pharmaceutically acceptable salt thereof and, optionally, the modulator of the Wnt/β-catenin pathway, such as an inhibitor of GSK-3 activity, or a pharmaceutically acceptable salt thereof.

The compounds of the present invention can be administered orally, parenterally, for example, subcutaneously, intravenously, intramuscularly, intraperitoneally, by intranasal instillation, by inhalation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may also be administered directly to the airways in the form of a dry powder. For use as a dry powder, the compounds of the present invention may be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

Suitable powder compositions include, by way of illustration, powdered preparations of the active ingredients thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aspects of the present invention are potential new combination drug therapies for treatment of bipolar disorder. Lithium is currently used as an anti-manic agent for patients with bipolar disorder. One of the biological effects of lithium is inhibition of the kinase GSK-3β. Inhibition of GSK-3β activates the Wnt/β-catenin-TCF/LEF transcriptional pathway.

It might be possible to use lower doses of the components in the present combination therapy, reducing the risk of toxic side effects.

### EXAMPLES

### Example 1

### Assay Description to Monitor Wnt/β-catenin Signalling

To monitor Wnt/β-catenin signalling the beta-catenin activated reporter (BAR) system developed at the laboratory of Randall Moon, University of Washington, Seattle, Wash., was used. The BAR system contains a concatemer of twelve TCF (T-cell factor) response elements separated by unique five nucleotide linkers specifically designed to minimize recombination that can lead to loss of TCF binding sites. This series of TCF response elements is inserted upstream of Promega's minP minimal promoter completing a functional promoter that drives the transcription of firefly luciferase (pBARLS) along with a PGK promoter driving a puromycin selectable maker. These reporters were inserted between the LTRs (long terminal repeats) of a lentiviral transducing plasmid. Control reporters, found unresponsive BAR (fuBARS), constructed using the same strategy were also used. The pfuBARS reporter is identical to their respective parent reporter with the exception that each TCF DNA binding element contains a two-base substitution conferring a non-functional element. The essentially identical nature of the control reporters provides the most optimal experimental control, as well as allowing for identical lentiviral titer production when generated side by side with the responsive reporter.

Assays were performed in stable cell lines (human HEK-293; mouse HT22) containing the pBARLS reporter and HEK-293 cells containing the pfuBARLS control. Cells were plated, treated, and the luciferase activity measured in a 384-well plate. The volume of culture media in each well prior to measuring luciferase activity was 40 μL. After incubation with compounds, 10 μL of Promega Firefly luciferase reagent was added using a liquid dispenser and incubated for 10 minutes at room temperature. Total luminescence was read on an Envision (Perkin Elmer) plate reader. Data was expressed relative to baseline of adding DMSO alone. L-cell control and Wnt3a conditioned media (ATCC CRL-2648 and CRL-2647) were used as controls to stimulate the reporter in different cell lines.

To measure the effects of compounds on HDAC activity, a homogenous, fluorimetric deacetylase assay miniaturized to 384-well plate format was used. To perform this assay, a buffered solution containing a recombinant human deacetylase was transferred to a microtiter plate using a robotic plate-filler.

Following compound transfer, a second buffered solution was transferred containing trypsin and a class-specific, tripeptide substrate (AcK-AMC) terminating in an acetyl-lysine, which is amide-coupled to 7-amino-4-methylcoumarin (AMC). Following substrate hydrolysis by the deacetylase, trypsin cleaves the terminal amide bond releasing AMC. With an excitation/emission maximum of 350-380/440-460 nm, AMC fluorescence is captured by a multilabel plate reader. For a kinetic version of the assay, fluorescence measurements was recorded every 0.5-5 minutes over 60 minutes. Data were plotted on a well-by-well basis to measure the slope of increasing fluorescence over time, corresponding to enzymatic activity. HDAC enzymes used were commercially-available (BPS Biosciences San Diego, Calif.).

### Example 2

### Activation of TCF/LEF Transcription by MS-275, CI-994, and SAHA

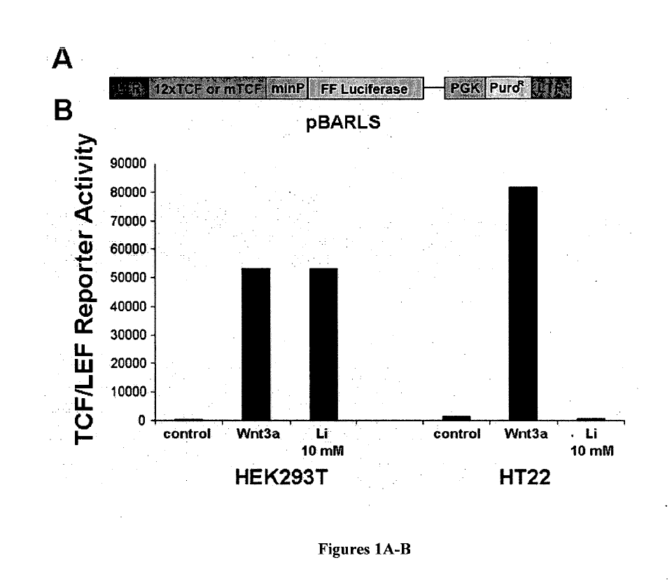

The results from pBARLS reporter in HEK293T cells are shown in FIG. 1. The activation of the pBARLS reporter by lithium was found to vary between different cell types, although in the case of HEK-293T and HT22 cells both were found capable of responding to Wnt3a conditioned media. The condition of lithium resistance was purposely exploited to identify compounds that could restore the sensitivity of cells to lithium. Compounds were tested then alone, in the presence of lithium, and Wnt3a.

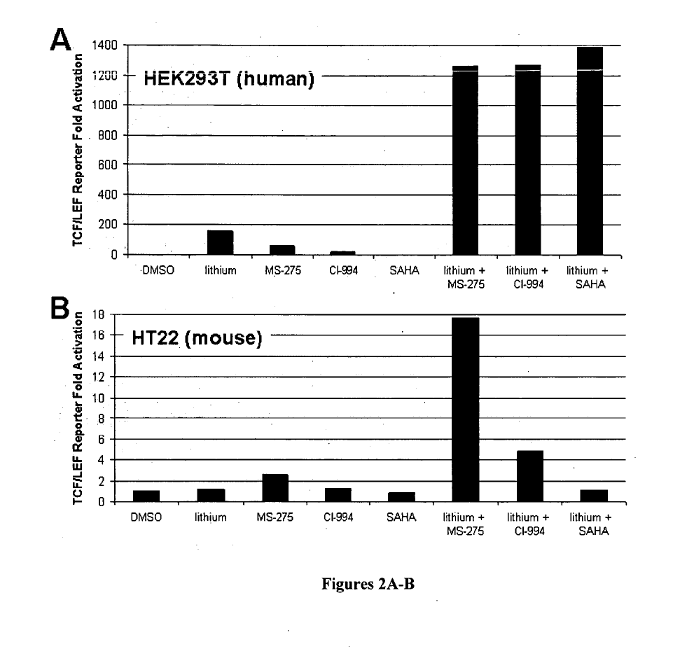

As shown in FIG. 2A, 24 hr treatment of HEK-293T cells with lithium, MS-275, CI-994, and SAHA activated transcription of the reporter with SAHA being weaker (˜2.5-fold). In the presence of lithium, all three compounds showed strong synergistic activation of transcription. Testing of these compounds in the pfuBARS control reporter in HEK-293T cells showed no activity of any of the compounds alone or in the presence of lithium. As shown in FIG. 2B, 24 hr treatment of HT22 cells with lithium, CI-994, and SAHA (25 uM) was unable to activate transcription whereas MS-275 was partially capable of activating transcription. However, in the presence of lithium, all three compounds showed strong synergistic activation of transcription similar to HEK-293T cells.

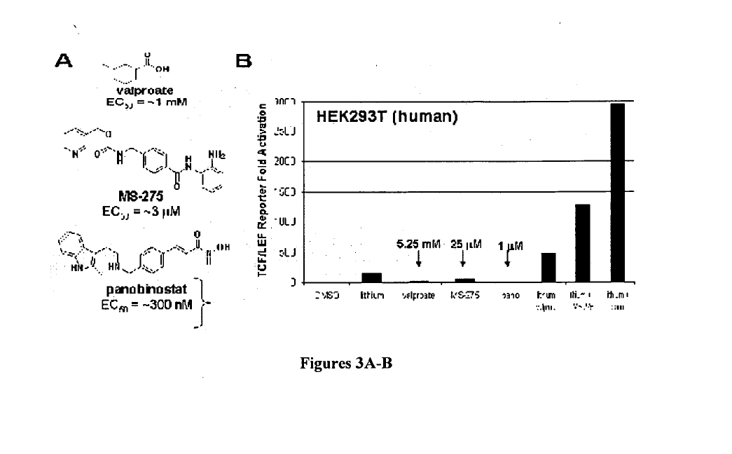

Dose responses of MS-275, as well as the bipolar drug valproate and the structural analog of SAHA, called panobinostat, that was found to be more potent than SAHA, revealed a range of potencies (see FIG. 3).

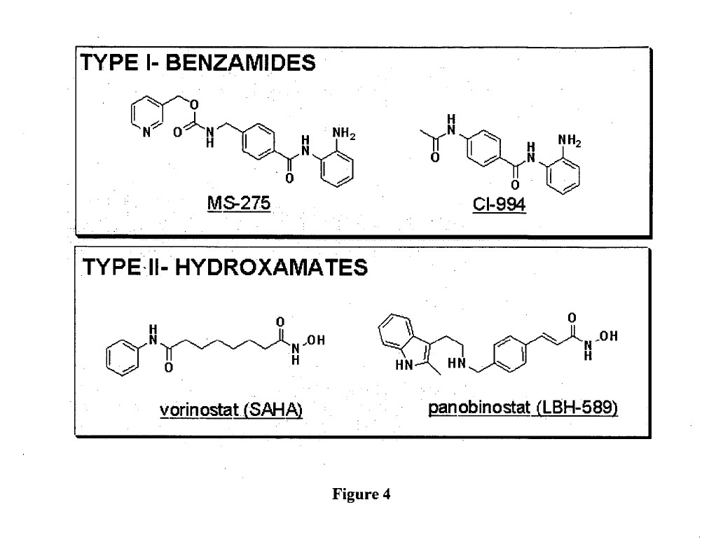

Based upon their structures, the compounds describe here can be grouped into two types of compounds (see FIG. 4). Without wishing to be bound by theory and without limitation regarding the nature of the molecular target(s) of the benzamides and hydroxamates as lithium potentiators, MS-275, CI-994, SAHA, and panobionstat are all known as inhibitors of histone deacetylases (HDACs). Since regulation of TCF/LEF transcription is known to involve chromatin remodeling and HDAC activity normally represses TCF/LEF transcription, it is plausible that HDACs are a relevant target although it remains unclear as to which class and isoforms of HDACs many be involved (see Billin, A., et al., Mol Cell Biol., 20(18):6882-90 (2000), which is hereby incorporated by reference in its entirety).

Acetylation of histone tails typically accompanies activation of transcription and renders chromatin in an ‘open’ conformation (see Kouzarides T., Cell 128(4):693-705 (2007), which is hereby incorporated by reference in its entirety). Deacetylation typically accompanies transcriptionally repressive events, rendering chromatin in a ‘closed’ conformation. A total of 18 genes have been identified that encode proteins with HDAC activity. These proteins have been classified into three subfamilies (HDAC I, II, and III) based upon DNA sequence and cofactor specificity. Class I and II HDACs (HDAC1-11) are metalloenzymes that rely on an active site zinc ion as part of the catalytic cycle to deacetylate the ε-amino group of lysine residues. Class III HDACs, also termed sirtuins, are also lysine deacetylases though this reaction is NAD⁺-dependent and results in NAD hydrolysis to 2′-O-acetyl-ADP-ribose and nicotinamide. HDACs function as part of large multiprotein complexes that are targeted to chromatin by DNA binding proteins. A number of biochemically purified HDAC-containing complexes have been characterized, including Sin3 complexes, CoREST complexes, and NuRD complexes. Recruitment of HDAC proteins to these complexes has been shown substantially to augment enzymatic function.

As summarized in Table 1 (below), whereas the hydroxamic acid SAHA is an inhibitor of class I and class II HDACs, MS-275 and CI-994 only inhibit class I HDACs. In comparison to valproate, a carboxylic acid used to treat bipolar disorder and previously reported as a class I and class II HDAC inhibitor (Gurvich N, et al., Cancer Res. 64(3):1079-86, which is hereby incorporated by reference in its entirety), MS-275 and CI-994 were found to share the same selectivity for class I HDACs but were much more potent with valproate's IC₅₀ being in the range of 27-130 μM for HDAC1/2/3 and no effect on HDAC5 and HDAC6.

TABLE 1
In vitro class I and class II histone deacetylase (HDAC) inhibition.

| | Class I | | | Class IIa | Class IIb |
|---|---|---|---|---|---|
| Compound | HDAC1 | HDAC2 | HDAC3 | HDAC5 | HDAC6 |
| valproate | 27 | 43 | 130 | >10 mM | >10 mM |
| MS-275 | 0.0776 | 0.1124 | 0.4217 | no inhib | no inhib |
| CI-994 | 0.170 | 0.400 | 0.690 | no inhib | no inhib |
| SAHA | 0.0046 | 0.0066 | 0.0166 | 14.1 | 0.0072 |

IC₅₀ (μM); avg n = 2 with class-specific substrates

Thus, the compounds discovered here define the relevance of class I HDACs to the modulation of Wnt/β-catenin signaling both in the case of Wnt3a and for lithium and expand the known classes of chelators that can potentiate TCF/LEF transcription and the activities of lithium to include aminobenzamides and cinnamic acid hydroxamates, both of which were not previously known.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A composition comprising:
a modulator of the Wnt/β-catenin pathway or a pharmaceutically acceptable salt thereof; and
a benzamide derivative, said benzamide derivative having the formula: 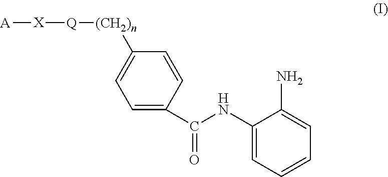
wherein,
A is H, a substituted or unsubstituted single-, fused- or multiple-ring aryl or heterocyclic ring systems, including saturated and unsaturated N-heterocycles, saturated and unsaturated S-heterocycles, and saturated and unsaturated O-heterocycles, saturated or unsaturated cyclic hydrocarbons, saturated or unsaturated mixed heterocycles;
X is absent, 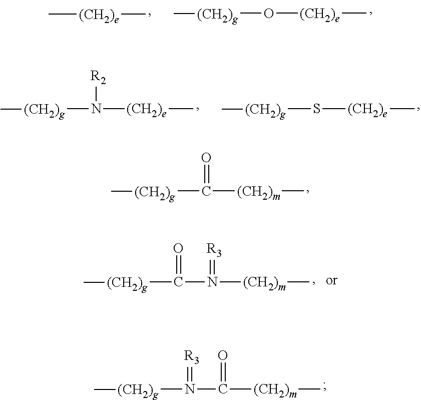
Q is 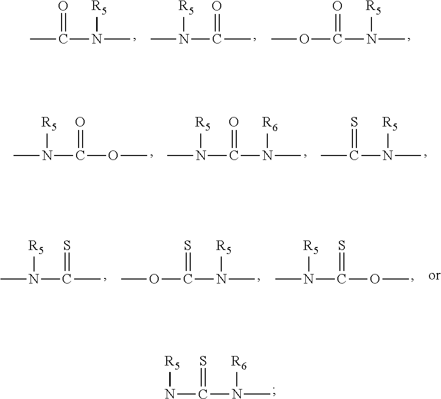
e is an integer from 1 to 4;
g is an integer from 0 to 4;
m is an integer from 0 to 4;
R₁ is H, halogen, hydroxyl, amino, nitro, cyano, C₁-C₄ alkyl, C₁-C₄ alkoxy, C₁-C₄ aminoalkyl, C₁-C₄ alkylamino, C₁-C₄ acyl, C₁-C₄ acylamino, C₁-C₄ alkylthio, C₁-C₄ perfluoroalkyl, C₁-C₄ perfluoroalkoxy, carboxyl, C₁-C₄ alkoxycarbonyl, aryl, or heterocycle or heteroaryl containing 1 to 5 heteroatoms selected from the group consisting of oxygen, nitrogen, and sulfur;
R₂ is H, substituted or unsubstituted C₁-C₄ alkyl, or 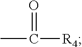
R₃ is H, or substituted or unsubstituted C₁-C₄ alkyl;
R₄ is H, substituted or unsubstituted C₁-C₄ alkyl, substituted or unsubstituted C₁-C₄ perfluoroalkyl, substituted or unsubstituted phenyl, or substituted or unsubstituted heterocycle;
R₅ and R₆ are each independently H, or substituted or unsubstituted C₁-C₄ alkyl; and
n is an integer from 0 to 4,
or a pharmaceutical salt thereof.

2. The composition of claim 1, further comprising: a pharmaceutically acceptable excipient or carrier.

3. The composition of claim 1, wherein the modulator of the Wnt/β-catenin pathway is a lithium compound or a pharmaceutically acceptable salt thereof.

4. The composition of claim 3, wherein the lithium compound is selected from the group consisting of lithium chloride, lithium citrate, lithium carbonate, lithium orotate, and mixtures thereof.

5. (canceled)

6. The composition of claim 1, wherein the modulator of the Wnt/β-catenin pathway is an inhibitor of GSK-3.

7. The composition of claim 1, wherein the modulator of the Wnt/β-catenin pathway is an inhibitor of GSK-3β.

8. The composition of claim 1, wherein the benzamide derivative has the formula: 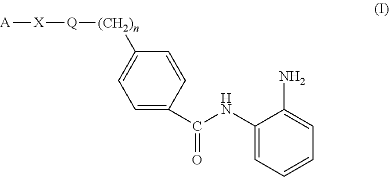
wherein,
A is H or an unsaturated N-heterocycle;
X is absent, 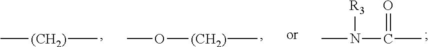
Q is 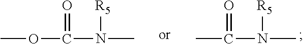
R₁, R₃, and R₅ are each H; and
n is 0 or 1,
or a pharmaceutical salt thereof.

9. The composition of claim 1, wherein the benzamide derivative has the formula:

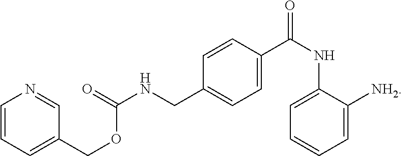 [SMILES: Nc1ccccc1NC(=O)c1ccc(CNC(=O)OCc2cccnc2)cc1]

10. The composition of claim 1, wherein the benzamide derivative has the formula:

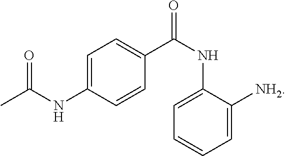 [SMILES: CC(=O)Nc1ccc(C(=O)Nc2ccccc2N)cc1]

11. A method of treating bipolar disorder in a subject, said method comprising:
administering a benzamide derivative, said benzamide derivative having the formula: 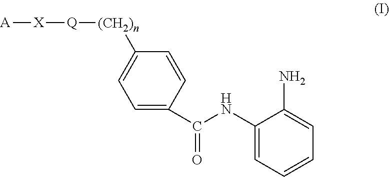
wherein,
A is H, a substituted or unsubstituted single-, fused- or multiple-ring aryl or heterocyclic ring systems, including saturated and unsaturated N-heterocycles, saturated and unsaturated S-heterocycles, and saturated and unsaturated O-heterocycles, saturated or unsaturated cyclic hydrocarbons, saturated or unsaturated mixed heterocycles;
X is absent, 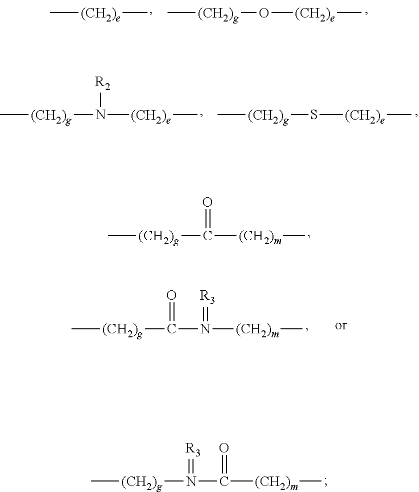
Q is 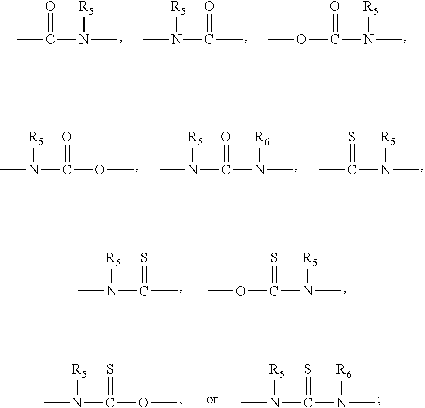
e is an integer from 1 to 4;
g is an integer from 0 to 4;
m is an integer from 0 to 4;
R₁ is H, halogen, hydroxyl, amino, nitro, cyano, C₁-C₄ alkyl, C₁-C₄ alkoxy, C₁-C₄ aminoalkyl, C₁-C₄ alkylamino, C₁-C₄ acyl, C₁-C₄ acylamino, C₁-C₄ alkylthio, C₁-C₄ perfluoroalkyl, C₁-C₄ perfluoroalkoxy, carboxyl, C₁-C₄ alkoxycarbonyl, aryl, or heterocycle or heteroaryl containing 1 to 5 heteroatoms selected from the group consisting of oxygen, nitrogen,
R₂ is H, substituted or unsubstituted C₁-C₄ alkyl, or 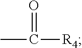
R₃ is H, or substituted or unsubstituted C₁-C₄ alkyl;
R₄ is H, substituted or unsubstituted C₁-C₄ alkyl, substituted or unsubstituted C₁-C₄ perfluoroalkyl, substituted or unsubstituted phenyl, or substituted or unsubstituted heterocycle;
R₅ and R₆ are each independently H, or substituted or unsubstituted C₁-C₄ alkyl; and
n is an integer from 0 to 4,
or a pharmaceutical salt thereof to the subject under conditions effective to treat a bipolar disorder.

12. (canceled)

13. The method of claim 11, wherein the benzamide derivative is administered in a composition further comprising:
a modulator of the Wnt/β-catenin pathway or a pharmaceutically acceptable salt thereof.

14. The method of claim 13, wherein the modulator of the Wnt/β-catenin pathway is a lithium compound selected from the group consisting of lithium chloride, lithium citrate, lithium carbonate, lithium orotate, and mixtures thereof.

15. (canceled)

16. The method of claim 13, wherein the modulator of the Wnt/β-catenin pathway is an inhibitor of GSK-3.

17. (canceled)

18. The method of claim 13, wherein the composition further comprises:
a pharmaceutically acceptable excipient or carrier.

19-22. (canceled)

23. A composition comprising:
a modulator of GSK-3 activity or a pharmaceutically acceptable salt thereof; and
a hydroxamic acid derivative, said hydroxamic acid derivative having the formula: 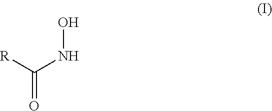
wherein,
R is C₂-C₆ alkyl, optionally substituted with substituents selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;
R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;
R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;
R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof,
or 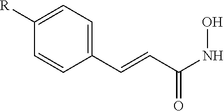
wherein,
R is selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;
R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;
R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;
R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof.

24. The composition of claim 23, further comprising:
a pharmaceutically acceptable excipient or carrier.

25. The composition of claim 23, wherein the modulator of GSK-3 activity is a lithium compound selected from the group consisting of lithium chloride, lithium citrate, lithium carbonate, lithium orotate, and mixtures thereof.

26. (canceled)

27. The composition of claim 23, wherein the modulator of GSK-3 activity is an inhibitor of GSK-3 β.

28. The composition of claim 23, wherein the hydroxamic acid derivative has the formula:

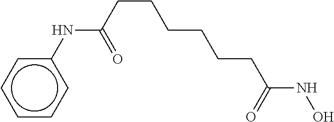

29. The composition of claim 23, wherein the hydroxamic acid derivative has the formula:

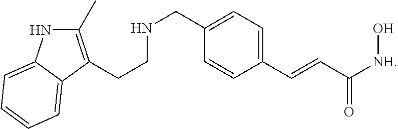 [SMILES: Cc1[nH]c2ccccc2c1CCNCc1ccc(/C=C/C(=O)NO)cc1]

30. A method of treating bipolar disorder in a subject, said method comprising:
administering a hydroxamic acid derivative, said hydroxamic acid derivative having the formula: 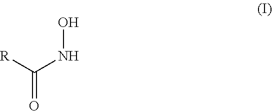
wherein,
R is C₂-C₆ alkyl, optionally substituted with substituents selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;
R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;
R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;
R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof,
or 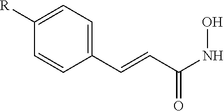
wherein,
R is selected from the group consisting of hydroxy, halogen, —NH₂, —NHR₁, —NR₁R₂, cyano, C(O)NHR₁, C₁-C₆ alkyl, C₂-C₆ alkenyl, C₂-C₆ alkynyl, C₄-C₇ cycloalkylalkyl, alkoxy, and R₃;
R₁ and R₂ are independently branched or unbranched C₁-C₆ alkyl, C₂-C₆ alkenyl, C₂-C₆ alkynyl, C₃-C₆ cycloalkyl, C₃-C₆ cycloalkenyl, or 5- to 6-membered monocyclic aryl, or heteroaryl containing 1-5 heteroatoms selected from the group consisting of oxygen, sulfur, and nitrogen, each one of R₁ and R₂ being optionally substituted with substituents selected from the group consisting of hydroxy, halogen, —NH₂, cyano, C₁-C₆ alkyl, C₂-C₆ alkenyl, C₂-C₆ alkynyl, and C₄-C₇ cycloalkylalkyl;
R₃ is a 5- to 6-membered monocyclic aryl, or heteroaryl containing 1-5 heteroatoms selected from the group consisting of oxygen, sulfur, and nitrogen, optionally substituted with a branched or unbranched C₁-C₆ alkyl, C₂-C₆ alkenyl, C₂-C₆ alkynyl, C₃-C₆ cycloalkyl, C₃-C₆ cycloalkenyl, or CH₂NHR₄;
R₄ is H, or branched or unbranched C₁-C₆ alkyl, C₂-C₆ alkenyl, C₂-C₆ alkynyl, C₃-C₆ cycloalkyl, C₃-C₆ cycloalkenyl, 5- to 6-membered monocyclic aryl, or heteroaryl containing 1-5 heteroatoms selected from the group consisting of oxygen, sulfur, and nitrogen, each optionally substituted with substituents selected from the group consisting of hydroxy, halogen, —NH₂, cyano, C₁-C₆ alkyl, C₂-C₆ alkenyl, C₂-C₆ alkynyl, C₄-C₇ cycloalkylalkyl, and indolyl optionally substituted with a halogen or C₁-C₆ alkyl; or a pharmaceutical salt thereof.

31. (canceled)

32. The method of claim 30, wherein the hydroxamic acid derivative is administered in a composition further comprising:
a modulator of GSK-3 activity or a pharmaceutically acceptable salt thereof.

33. The method of claim 32, wherein the modulator of GSK-3 activity is a lithium compound selected from the group consisting of lithium chloride, lithium citrate, lithium carbonate, lithium orotate, and mixtures thereof.

34. (canceled)

35. The method of claim 32, wherein the modulator of GSK-3 activity is an inhibitor of GSK-3 β.

36. The method of claim 32, wherein the composition further comprises:
a pharmaceutically acceptable excipient or carrier.

37-39. (canceled)
